(19) United States
(12) Patent Application Publication
(10) Pub. No.: US 2021/0169454 A1
(43) Pub. Date: Jun. 10, 2021

(54) ULTRASONIC ANALYSIS APPARATUS, ULTRASONIC ANALYSIS METHOD, AND ULTRASONIC ANALYSIS PROGRAM

(71) Applicant: FURUNO ELECTRIC CO., LTD., Nishinomiya-City (JP)

(72) Inventor: Tatsuo ARAI, Takarazuka (JP)

(73) Assignee: FURUNO ELECTRIC CO., LTD., Nishinomiya-City (JP)

(21) Appl. No.: 17/182,103

(22) Filed: Feb. 22, 2021

Related U.S. Application Data
(63) Continuation of application No. PCT/JP2019/027884, filed on Jul. 16, 2019.

(30) Foreign Application Priority Data
Aug. 22, 2018 (JP) 2018-155356

(51) Int. Cl. A61B 8/08; A61B 8/00

(52) U.S. Cl. CPC A61B 8/5223; A61B 8/463; A61B 8/4272

(57) ABSTRACT

The purpose is to evaluate the condition of a muscle in a relatively simple constitution without accompanying exposure. An ultrasonic analysis apparatus comprises an interface configured to receive signals from a transmitter/receiver which receives ultrasonic waves transmitted from a plurality of mutually different positions on a surface of a body toward a muscle inside the body and reflected from inside the body, processing circuitry configured to generate ultrasonic images corresponding to the respective positions respectively based on the signals inputted from the interface, to stitch the ultrasonic images at the respective positions to generate a stitched image, to set a region of interest for at least the stitched image and the ultrasonic image, and to calculate an index related to the muscle from at least one of the respective images corresponding to the region of interest.

### CROSS-REFERENCE TO RELATED APPLICATION(S)

This application is a continuation of PCT International Application No. PCT/JP2019/027884, which was filed on Jul. 16, 2019, and which claims priority to Japanese Patent Application Ser. No. 2018-155356 filed on Aug. 22, 2018, the entire disclosures of each of which are herein incorporated by reference for all purposes.

### TECHNICAL FIELD

The present invention relates to an ultrasonic analysis apparatus, an ultrasonic analysis method and an ultrasonic analysis program for analyzing the inside of a body by ultrasonic waves.

### BACKGROUND

The decline of muscle with aging is manifested not only as a decrease in muscle mass but also as a decrease in muscle quality. There is no clear definition of muscle quality, but the main index includes increased intramuscular fat, fibrosis, fast/slow muscles, pennation angle, aerobic capacity, and neuromuscular junction functioning. Among these, intramuscular fat is the index of the muscle quality which is related to the motor function degeneracy of the elderly and needs to be measured. An increase in the intramuscular fat leads to a decrease in muscle strength that can be exerted without a decrease in muscle mass.

Conventionally, a technique for measuring the muscle quality from a muscle cross-sectional image acquired by a CT (Computed Tomography) apparatus or an MRI (Magnetic Resonance Imaging) apparatus has been used. In non-patent document 1, since there is a significant correlation between the skeletal muscle average CT value and the intramuscular fat amount obtained by muscle biopsy, it is shown that the skeletal muscle average CT value is effective as an evaluation index of the muscle quality.

### REFERENCE DOCUMENTS OF CONVENTIONAL ART

### Non-Patent Document 1

Goodpaster B H, et al, “Skeletal muscle attenuation determined by computed tomography is associated with skeletal muscle lipid content”, J Appl Physiol (1985), 2000 July, 89 (1), 104-10 (1), 104-110

However, CT and MRI systems are complex, expensive, and involve exposure to possibly harmful radiation. In addition, since there is no dedicated software for image analysis, it cannot be handled by general engineers.

An object of the present invention is to provide an ultrasonic analysis apparatus for evaluating the state of a muscle with a relatively simple configuration without being exposed to radiation.

### SUMMARY

An ultrasonic analysis apparatus according to the present invention is characterized by comprising: an interface module configured to receive signals from a transmitter/receiver which receives ultrasonic waves transmitted from a plurality of mutually different positions on the surface of a body toward muscles inside the body and reflected from inside the body; an ultrasonic image generating module configured to generate ultrasonic images corresponding to the respective positions based on the signals inputted from the interface module; an image compositing module configured to stitch the ultrasonic images at the respective positions to generate a stitched image; a region setting module configured to set a region of interest for at least the stitched image of the stitched images and the ultrasonic images; and an index calculation module configured to calculate an index related to the muscle from at least one of the images corresponding to the region of interest.

An ultrasonic analysis method according to the present invention is characterized by comprising the steps of: receiving a signal from a transmitter/receiver which receives ultrasonic waves transmitted from a plurality of mutually different positions on a surface of a body toward muscles from inside the body and reflected therein; respectively generating ultrasonic images corresponding to the respective positions based on the received signal; stitching the ultrasonic images at the respective positions to generate a stitched image; setting a region of interest for at least the stitched image of the stitched image and the ultrasonic image; and calculating an index related to the muscle from at least one of the respective images corresponding to the region of interest.

An ultrasonic analysis program according to the present invention is a non-transitory computer-readable recording medium storing a program causing a processor of an ultrasound analysis apparatus to execute processing, the processor configured to control operation of the ultrasound analysis apparatus, the processing comprising: receiving input of signals from a transmitter/receiver which receives ultrasonic waves transmitted from a plurality of mutually different positions on a surface of a body toward muscles inside the body and reflected from inside the body, generating ultrasonic images corresponding to the respective positions respectively based on inputted signals, stitching the ultrasonic images at the respective positions to generate a stitched image, setting a region of interest for at least the stitched image of the stitched images and the ultrasonic images, and calculating an index related to the muscle from at least one of the images corresponding to the region of interest.

According to the present invention, the state of the muscle can be evaluated with a relatively simple configuration and without being exposed to radiation.

### BRIEF DESCRIPTION OF THE DRAWINGS

The illustrated embodiments of the subject matter will be best understood by reference to the drawings, wherein like parts are designated by like numerals throughout. The following description is intended only by way of example, and simply illustrates certain selected embodiments of devices, systems, and processes that are consistent with the subject matter as claimed herein:

FIG. 1 is a block diagram of an ultrasound analysis system according to an embodiment of the present invention;

FIGS. 2A and 2B illustrate an operational aspect of an ultrasound probe;

FIGS. 3A and 3B are diagrams for explaining an operation mode of a linear scan mode and a sector scan mode;

FIG. 4 is a functional block diagram of a signal processing module;

FIGS. 5A-5D illustrate the generation of a linear scan image;

FIGS. 6A and 6B illustrate the generation of a sector scan image;

FIG. 7 is an example of a stitched image of a thigh;

FIGS. 8A-8D are diagrams for explaining the setting of a region of interest in a stitched image;

FIGS. 9A and 9B are examples of an ultrasound image in which a region of interest is set;

FIGS. 10A and 10B are examples of a region of interest extracted from an ultrasound image;

FIGS. 11A-11D are graphs showing a relationship between a brightness of a region of interest and an image number;

FIGS. 12A and 12B are histograms representing a brightness distribution;

FIG. 13 is a graph showing the relationship between the brightness of the region of interest and the image number;

FIG. 14 shows a linear scan image and a two-dimensional fast Fourier transformed image of it;

FIG. 15 is a flow chart showing a flow of processing in an ultrasonic analysis method according to an embodiment of the present invention; and

FIG. 16 is a diagram for explaining another example of setting a region of interest in a stitched image.

### DETAILED DESCRIPTION

Hereinafter, preferred embodiments of the present invention will be described with reference to the drawings. The following embodiments illustrate a method and apparatus for embodying the technical idea of the present invention, and the technical idea of the present invention is not limited to the following. The technical idea of the present invention can be modified in various ways within the scope of the claims.

Embodiments of the present invention will now be described in detail with reference to the accompanying drawings. In the following description and the drawings, the same reference numerals indicate the same or similar components, and therefore redundant description of the same or similar components is omitted. In the following description, the quadriceps femoris is the analysis subject of muscle quality, but the type of muscle is not particularly limited in the present invention.

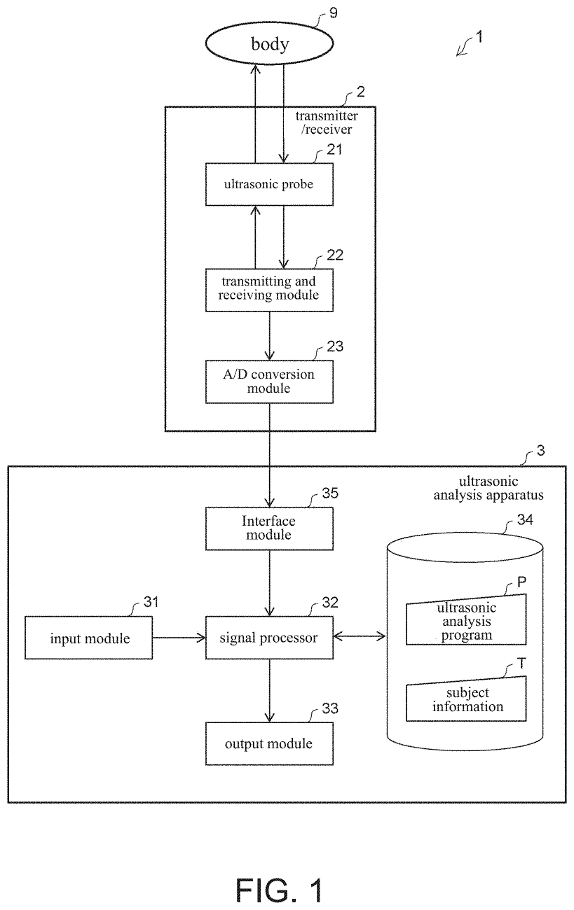

FIG. 1 is a block diagram of an ultrasonic analysis system 1 according to an embodiment of the present invention. The ultrasonic analysis system 1 includes a transmitter/receiver 2 and an ultrasonic analysis apparatus 3 connected to the transmitter/receiver 2.

The transmitter/receiver 2 is a device for transmitting ultrasonic waves toward muscles inside a body 9 and receiving echo signals reflected from inside the body 9. The transmitter/receiver 2 includes an ultrasonic probe 21, a transmitting and receiving module 22, and an A/D conversion module 23.

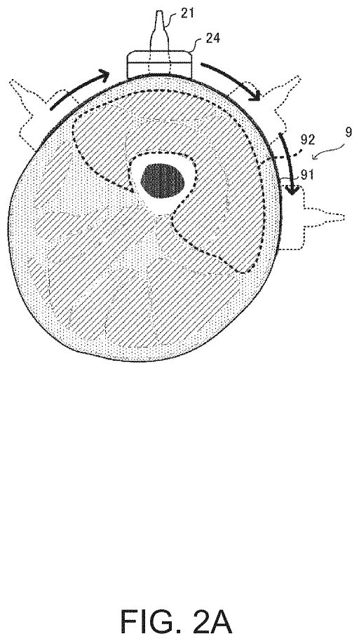

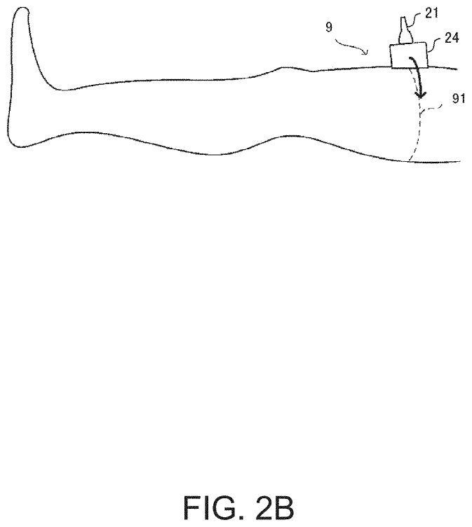

As shown in FIG. 2A, the ultrasonic probe 21 is a device for transmitting ultrasonic waves from a plurality of different positions on a surface of the body 9 toward the inside of the body 9 and receiving the ultrasonic waves reflected from the inside of the body 9. In this embodiment, the ultrasonic probe 21 is configured to be gripped and moved by a user, and an ultrasonic transmission/reception surface is provided on a lower end surface of the ultrasonic probe 21. An ultrasonic array sensor, constituted by arranging a plurality of ultrasonic vibrators in a line, is arranged on the ultrasonic transmission/reception surface. When acquiring a tomographic image of the body 9 (or a cross-sectional image), the user makes contact between the body 9 and the ultrasonic transmission/reception surface of the ultrasonic probe 21, and moves the ultrasonic probe 21 along the surface of the body 9 (scans with the ultrasonic probe 21). Meanwhile, the ultrasonic probe 21 intermittently transmits ultrasonic waves from the ultrasonic transmission/reception surface toward the inside of the body 9, and receives the ultrasonic waves reflected from the inside of the body 9 on the ultrasonic transmission/reception surface. Thus, the ultrasonic probe 21 outputs an electric signal (echo signal) indicating the received ultrasonic wave.

In an embodiment, as shown in FIGS. 2A and 2B, the ultrasonic probe 21 is attached to a probe adapter 24, and a contacting surface of the probe adapter 24 contacts on the surface of a thigh 91 of the body 9. In this state, the user moves the ultrasonic probe 21 and the probe adapter 24 along an outer periphery of a transverse section of the thigh 91 that is to be imaged. While the ultrasonic probe 21 is moved along the surface of the thigh 91, ultrasonic waves are transmitted at predetermined time intervals from the plurality of ultrasonic vibrators of the ultrasonic probe 21. Thus, the ultrasonic probe 21 transmits ultrasonic waves from different positions toward a quadriceps femoris 92 a plurality of times.

The ultrasonic probe 21 or the probe adapter 24 may be provided with an inclination sensor. The inclination sensor detects an inclination of the ultrasonic probe 21 with respect to a vertical direction, that is, a direction in which the ultrasonic transmission/reception surface is facing. Further, although an example in which the ultrasonic probe 24 to which the probe adapter 24 is attached, alternatively, the ultrasonic image may be acquired by moving only the ultrasonic probe 21 without using the probe adapter 24.

The ultrasonic probe 21 is operable in two modes, a linear scan mode for acquiring a linear scan image and a sector scan mode for acquiring a sector scan image.

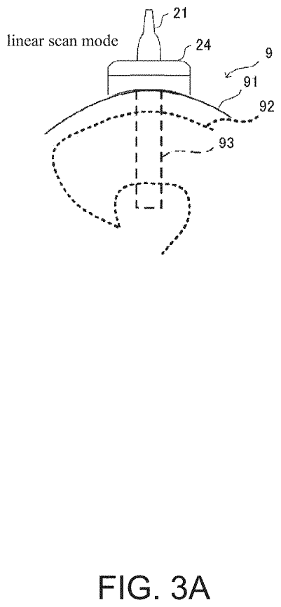

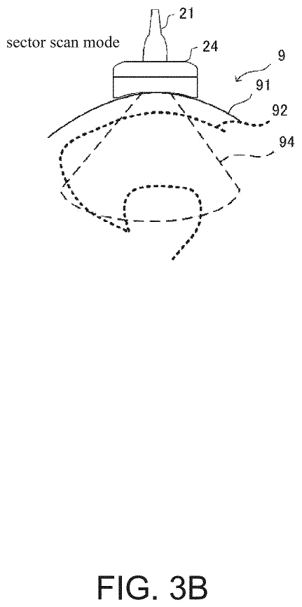

When the ultrasonic probe 21 is operated in the linear scan mode, as shown in FIG. 3A, the ultrasonic wave transmitted from the ultrasonic transmission/reception surface is directed toward a zonal region 93. Therefore, when the ultrasonic probe 21 is driven in the linear scan mode, the ultrasonic analysis apparatus 3 can generate an ultrasonic image in which the zonal region 93 is clearly imaged.

When the ultrasonic probe 21 is operated in the sector scan mode, as shown in FIG. 3B, the ultrasonic wave transmitted from the ultrasonic transmission/reception surface is directed toward a fan-shaped region 94. Therefore, when the ultrasonic probe 21 is driven in the sector scan mode, the ultrasonic analysis apparatus 3 can generate an ultrasonic image obtained by imaging the fan-shaped region 94.

In the present embodiment, the ultrasonic probe 21 operates while alternately repeating the linear scan mode and the sector scan mode, and acquires multiple linear scan images and sector scan images, for example, 200 images are acquired during the linear scan mode and the sector scan mode, thus a total of 400 images are acquired. The number of the images is only one example, and is set to an arbitrary value. The number may be determined according to the operation and the condition of the subject. The ultrasonic probe 21 may be configured to acquire either a linear scan image or a sector scan image at a given time.

The transmitter/receiver 2 shown in FIG. 1 forms a waveform of a carrier wave that constitutes a frequency of an ultrasonic band into a pulse shape to generate a transmission pulse, and outputs the generated transmission pulse to the ultrasonic probe 21. Thus, ultrasonic waves are transmitted from the plurality of ultrasonic vibrators of the ultrasonic probe 21 in a depth direction of the body 9. The transmitting and receiving module 22 receives the echo signal received by the plurality of ultrasonic vibrators of the ultrasonic probe 21 from the inside of the body 9.

The A/D conversion module 23 converts the echo signal transmitted from the transmitting and receiving module 22 from an analog format to a digital format, and transmits the converted echo signal to the ultrasonic analysis apparatus 3.

The ultrasonic analysis apparatus 3 includes an input module 31, a signal processing module 32 (also referred as “processing circuitry”, described later), and an output module 33.

In the present embodiment, the ultrasonic analysis apparatus 3 is constituted by a known personal computer, and includes, as a hardware configuration, a processor (not shown) such as a CPU for performing data processing, a memory (not shown) used by the processor in a work area for data processing, a recording module 34 for recording processing data, a bus (not shown) for transmitting data between each module, and an interface module (hereinafter referred to as I/F module 35) for inputting/outputting data to/from an external apparatus. As an optional function, the ultrasonic analysis apparatus 3 can be connected to an external server via a network such as the Internet.

The input module 31 receives input of an operation from a user. For example, the input module 31 may be a keyboard, a mouse, a touch panel, or the like.

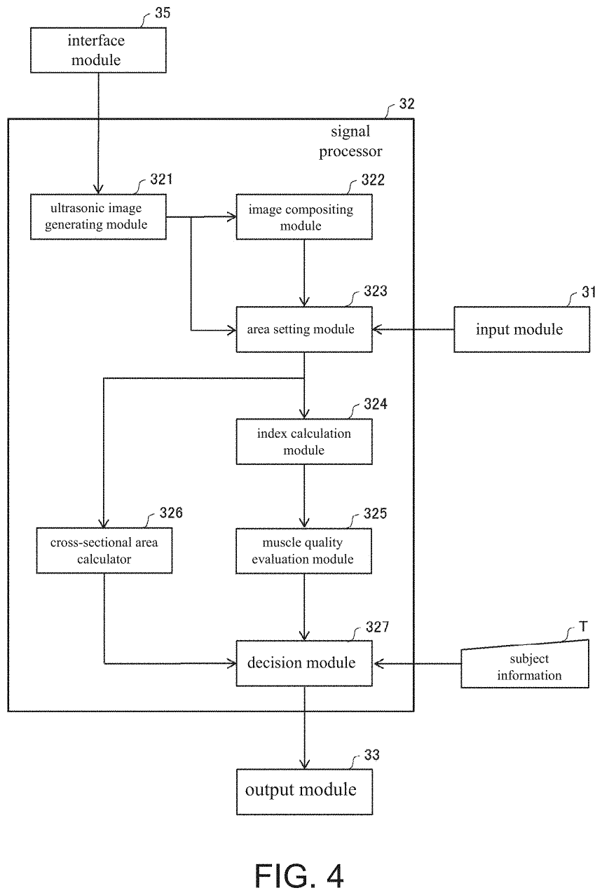

The signal processing module 32 (or “processing circuitry”) is a functional block that receives an echo signal converted into the digital format from the transmitter/receiver 2 and performs various arithmetic processing functions such as calculation of an index corresponding to a muscle quality of the quadriceps femoris 92 to be analyzed. The signal processing module 32 may be implemented in hardware by a logic circuit formed on an integrated circuit, but in this embodiment, the signal processing module 32 is implemented in software by the processor executing an ultrasonic analysis program P previously recorded in the recording module 34 or the memory. The ultrasonic analysis program P may be installed in the ultrasonic analysis apparatus 3 via a network. Alternatively, the ultrasonic analysis program P may be installed in the ultrasonic analysis apparatus 3 by causing the ultrasonic analysis apparatus 3 to read a computer-readable non-temporary tangible recording medium, such as a CD-ROM, in which the ultrasonic analysis program P is recorded. Details of the signal processing module 32 will be described later with reference to FIG. 4.

The output module 33 outputs a calculation result of the signal processing module 32. By way of example, the output module 33 can be configured with a monitor, a printer, or the like.

The I/F module 35 receives a signal (the converted echo signal) from the transmitter/receiver 2.

FIG. 4 is a functional block diagram of the signal processing module 32. The signal processing module 32 includes an ultrasonic image generating module 321, an image compositing module 322, an area setting module 323, an index calculation module 324, a muscle quality evaluation module 325, a cross-sectional area calculator 326, and a decision module 327.

The ultrasonic image generating module 321 generates an ultrasonic image of the inside of the body 9 based on the echo signal input from the I/F module 35. As described above, the transmitter/receiver 2 transmits ultrasonic waves a plurality of times while alternately repeating the linear scan mode and the sector scan mode, and receives the echo signals reflected from inside the body 9. Therefore, each time the transmitter/receiver 2 receives the echo signal, the I/F module 35 receives the converted echo signal. In response to this, the ultrasonic image generating module 321 generates a linear scan image as an ultrasonic image from the echo signal received in the linear scan mode, and generates a sector scan image as an ultrasonic image from the echo signal received in the sector scan mode. In the present embodiment, the ultrasonic image generating module 321 generates multiple linear scan images and multiple sector scan images, for example, 200 linear scan images and 200 sector scan images, thus, a total of 400 ultrasonic images.

The ultrasonic image generating module 321 may correct the deformation caused by the pressing of the ultrasonic probe 21 against the cross section of the body 9 in the ultrasonic image, based on a force of the ultrasonic probe 21 pressing the body 9 and a position and direction of the ultrasonic probe 21.

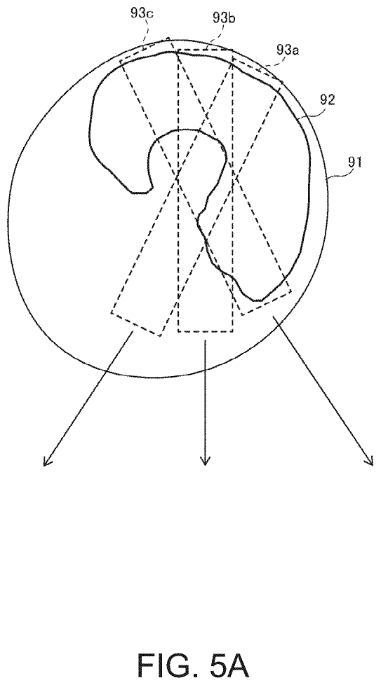

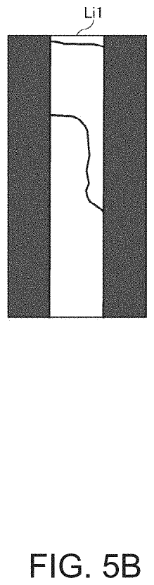

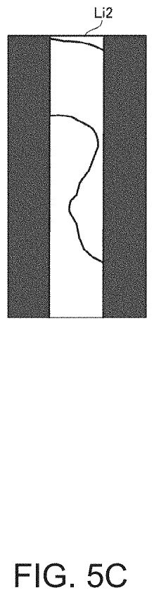

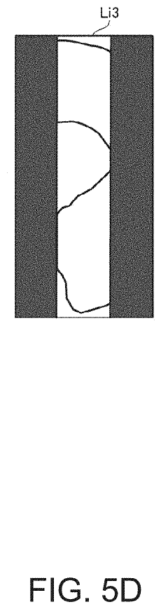

FIG. 5 is a diagram for explaining generation of a linear scan image. FIG. 5A is a partial sectional view of the thigh 91, and the thigh 91 is divided into the quadriceps femoris 92 and other portions for convenience. In the linear scan mode, since the ultrasonic wave is directed to the zonal region 93, a real image region of the linear scan image also becomes the zonal region 93. For example, when the ultrasonic wave is directed to a zonal region 93a, the linear scan image Li1 as shown in FIG. 5B is generated, when the ultrasonic wave is directed to a zonal region 93b, the linear scan image Li2 as shown in FIG. 5C is generated, and when the ultrasonic wave is directed to a zonal region 93c, the linear scan image Li3 as shown in FIG. 5D is generated.

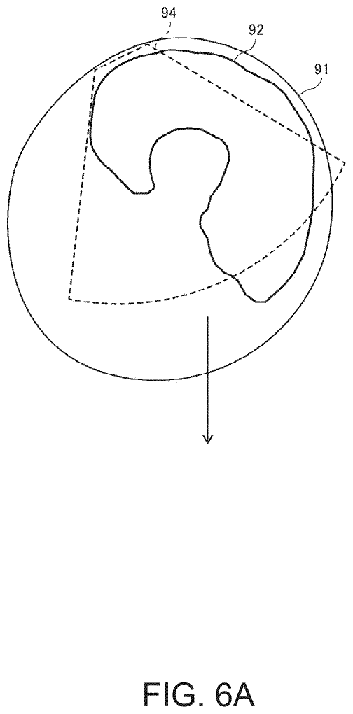

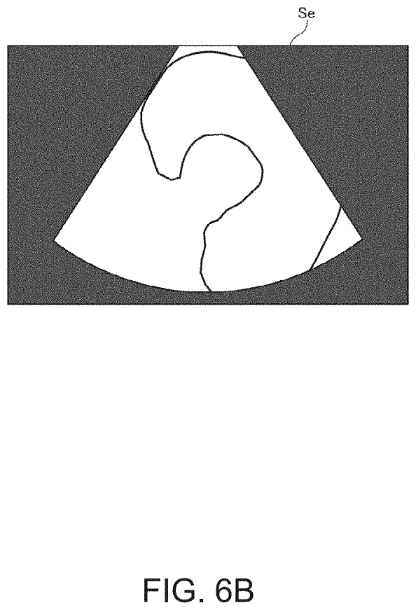

FIG. 6 is a diagram for explaining generation of a sector scan image. FIG. 6A is a partial sectional view of the thigh 91, and similarly to FIG. 5A, the thigh 91 is divided into the quadriceps femoris 92 and other portions for convenience. In the sector scan mode, since the ultrasonic wave is directed to the fan-shaped region 94, a real image region in the sector scan image Se is fan-shaped as shown in FIG. 6B.

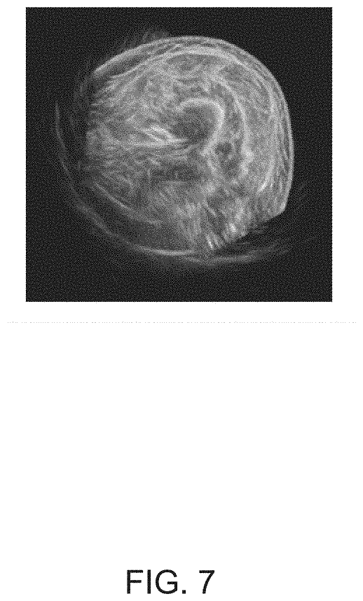

The image compositing module 322 stitches a plurality of ultrasonic images (linear scan image and sector scan image) generated by the ultrasonic image generating module 321 to generate a stitched image. The method for stitching the ultrasonic image is not particularly limited, and any known method can be used. For example, in the case of generating a stitched image by the method described in WO2017/010193A1, the image compositing module 322 detects and matches a feature amount between regions included in each of the plurality of ultrasonic images, thereby determining a position at which the plurality of ultrasonic images overlap. When the ultrasonic probe 21 or the probe adapter 24 is provided with the inclination sensor, the image compositing module 322 rotates each ultrasonic image on the basis of a detection angle obtained from the inclination sensor, and performs matching on the basis of the rotated ultrasonic image. Thus, a rotation angle of each ultrasonic image can be accurately corrected, and an overlapped position of a fragment image can be determined with higher accuracy. FIG. 7 is an example of a stitched image of the thigh 91.

The area setting module 323 shown in FIG. 4 is a functional block for setting a region of interest (ROI: Region of Interest) for at least one of the stitched image and the ultrasonic image. In the present embodiment, the area setting module 323 is a functional block that sets a region of interest for the stitched image and sets a region of interest for the ultrasonic image corresponding to the region of interest of the stitched image before the generation of the stitched image.

The area setting module 323 may set one region of interest for the entire quadriceps femoris 92 included in the stitched image, or may evaluate the muscle qualities of each of a rectus femoris, vastus lateralis, vastus intermedius, and vastus medialis constituting the quadriceps femoris 92 in order to accurately evaluate the muscle qualities of the quadriceps femoris 92. Therefore, in this embodiment, the area setting module 323 sets a region of interest for each of the rectus femoris, vastus lateralis, vastus intermedius, and vastus medialis.

The area setting module 323 sets the region of interest in the stitched image in response to a user's operation via the input module 31. Hereinafter, a specific example of setting a region of interest in the stitched image shown in FIG. 7 will be described.

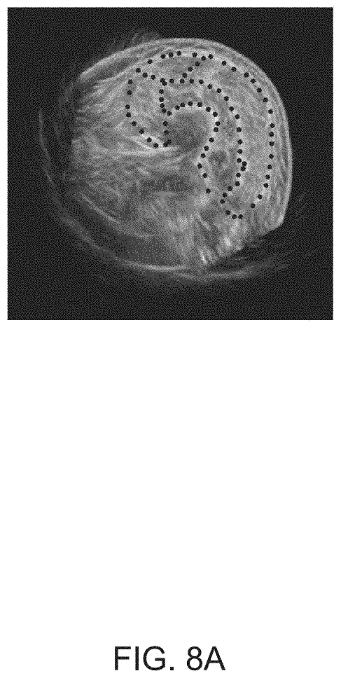

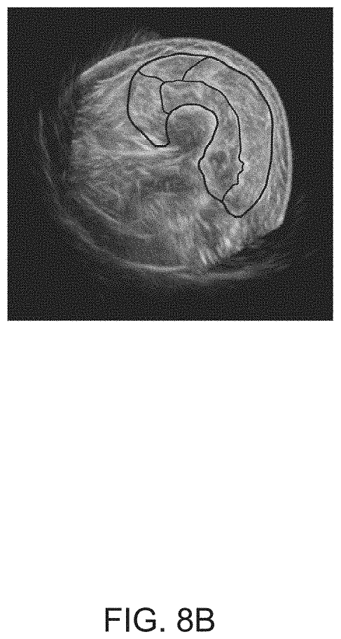

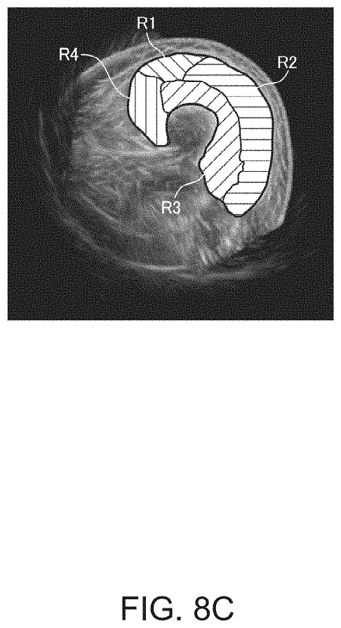

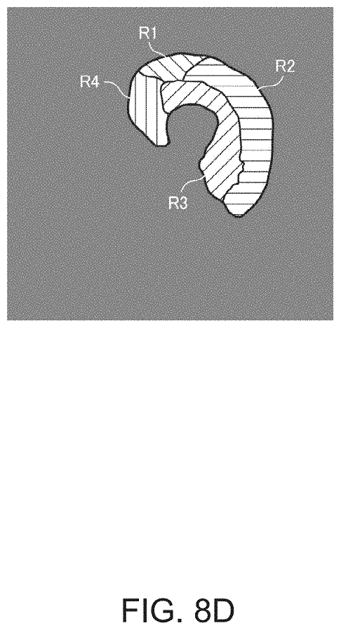

The user operates the mouse or the like while viewing the stitched image shown in FIG. 7, and as shown in FIG. 8A, hits a point at the boundary position of the rectus femoris, vastus lateralis, vastus intermedius, and vastus medialis. In this case, a color of the point may be different depending on a type of the adjacent tissue.

The points shown in FIG. 8A may be automatically set by a method for automatically detecting the boundaries of the rectus femoris, vastus lateralis, vastus intermedius, and vastus medialis.

Subsequently, as shown in FIG. 8B, in response to a user's instruction via the input module 31, points of the same color are connected to each other, whereby the signal processing module 32 creates a boundary line. Thus, as shown in FIG. 8C, the quadriceps femoris 92 is divided into 4 regions such as the rectus femoris, the vastus lateralis, the vastus intermedius, and the vastus medialis, and the area setting module 323 sets 4 regions as regions of interest R1 to R4, respectively. Thereafter, as shown in FIG. 8D, the area setting module 323 extracts a mask image (masked image) comprising the set regions of interest R1 to R4 from the stitched image. As described above, setting of the region of interest for the stitched image is completed.

The regions of interest R1 to R4 are divided by hatching, a type of hatching is independent of brightness, which will be described later.

Further, the area setting module 323 sets a region of interest corresponding to the region of interest of the stitched image also for the ultrasonic image before the stitch. In the present embodiment, the area setting module 323 overlaps the mask images composed of the regions of interest R1 to R4 extracted from the stitched image with the ultrasonic images. At this time, the mask image is rotated according to the rotation angle of each ultrasonic image. Thus, the area setting module 323 can specify the boundary of the region of interest in each ultrasonic image and also set the region of interest for each ultrasonic image.

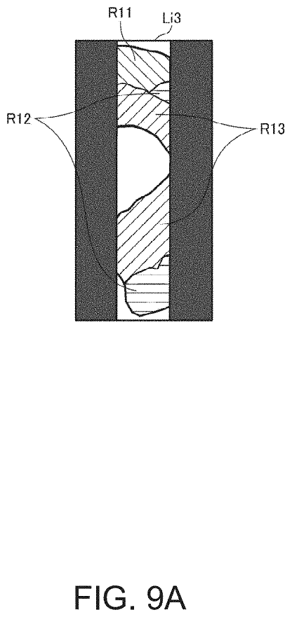

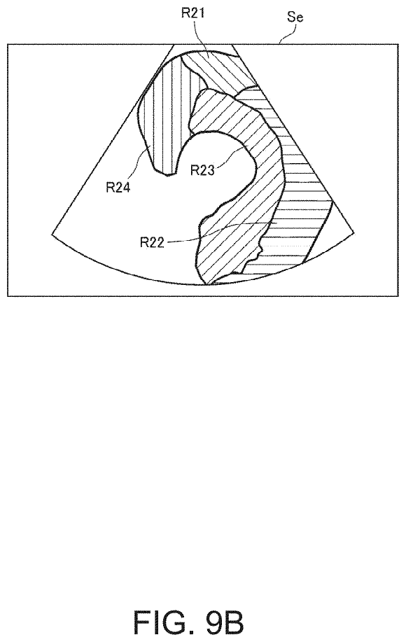

In an example, for the linear scan image Li3 shown in FIG. 5D, the regions of interest R11, R12 and R13 included in the linear scan image Li3 are set as shown in FIG. 9A. For the sector scan image Se shown in FIG. 6B, regions of interest R21, R22, R23 and R24 included in the sector scan image Se are set as shown in FIG. 9B.

In the following description, the region of interest (For example, regions of interest R11 to R13 and R21 to R24) set in the ultrasonic image before synthesis is sometimes referred to as “secondary region of interest”.

The index calculation module 324 shown in FIG. 4 calculates an index related to the muscle from at least one of the images (stitched image and ultrasonic image) corresponding to the region of interest. In the present embodiment, the index calculation module 324 calculates an index corresponding to the muscle quality of the muscle based on the image (here, it is an ultrasonic image, more specifically, a linear scan image or a sector scan image) corresponding to the secondary region of interest set for the ultrasonic image before synthesis. Since the brightness of the image of the muscle increases as the intramuscular fat content increases, the index calculation module 324 calculates the average value of the brightness of the image corresponding to the region of interest as an index. A specific example of the calculation method of the index is described below.

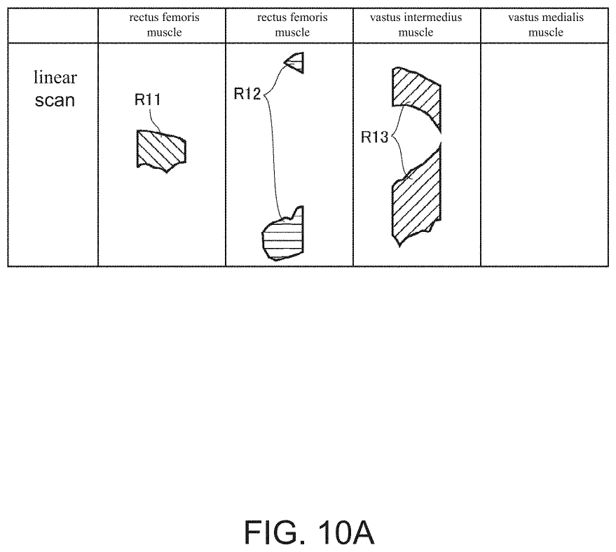

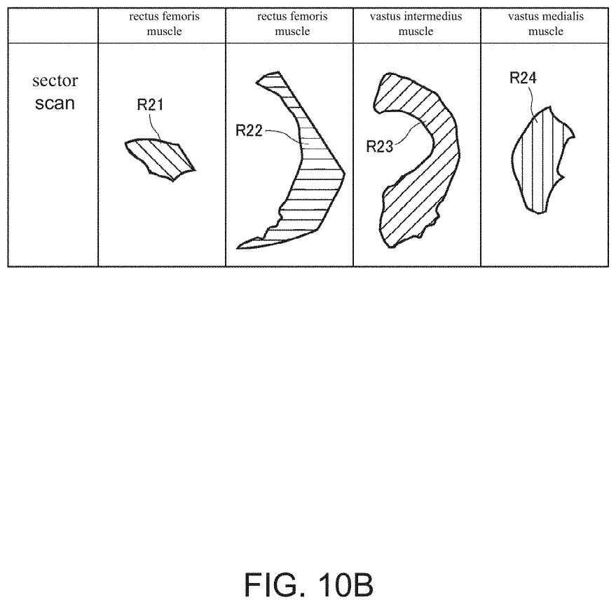

First, the index calculation module 324 extracts the secondary region of interest set in each ultrasonic image. For example, from the linear scan image Li3 shown in FIG. 5D, secondary regions of interest R11, R12, and R13 corresponding to the rectus femoris, vastus lateralis, and vastus intermedius, respectively, are extracted as shown in FIG. 10A. Further, from the sector scan image Se shown in FIG. 6B, secondary regions of interest R21, R22, R23, and R24 corresponding to the rectus femoris, vastus lateralis, vastus intermedius, and vastus medialis, respectively, are extracted as shown in FIG. 10B. The index calculation module 324 performs the extraction process for all the linear scan images and the sector scan images.

Since there is a line (Lines of fascia within bones and the vastus lateralis) with high brightness in the region of interest, an outer peripheral edge of the region of interest may be removed (For example, a few pixels) for extraction. For the removal of high brightness lines, for example, line enhancement image filters, binarization, and high brightness line detection by the Dijkstra's algorithm, can be used.

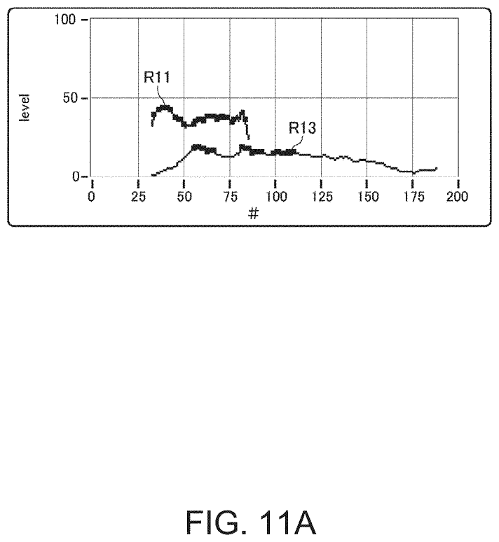

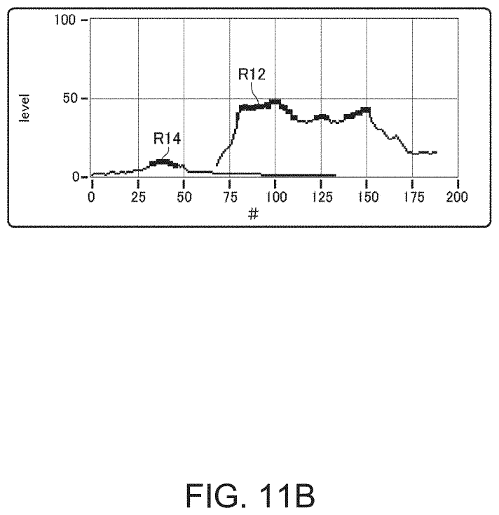

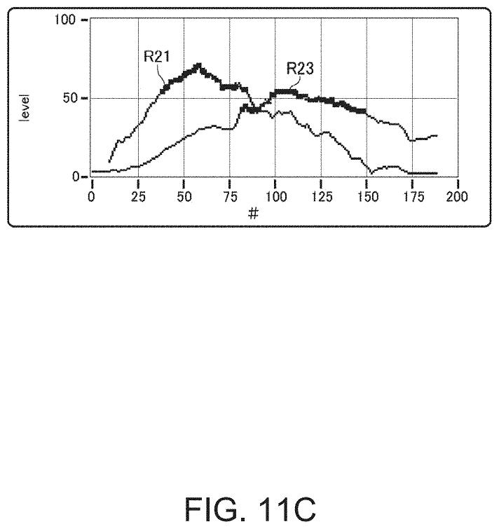

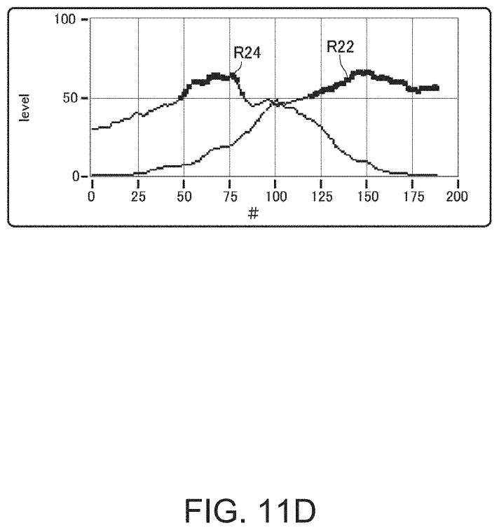

Subsequently, the index calculation module 324 calculates the brightness of each region of interest. FIGS. 11A and 11B are graphs showing the brightness of the secondary regions of interest R11 to R13 extracted from the 200 linear scan images, and FIGS. 11C and 11D are graphs showing the brightness of the secondary region of interests R21 to R24 extracted from the 200 sector scan images. In each graph, the horizontal axis represents a number assigned to the acquisition order of the linear scan image or the sector scan image, and the vertical axis represents the brightness of the secondary region of interest. In addition, a portion where the brightness is 70% or more of the maximum brightness of the secondary region of interest is indicated by a thick line.

The index calculation module 324 calculates an average value of brightness indicated by a thick line for each secondary region of interest, and outputs the calculated average value as an index corresponding to the muscle quality. In calculating the index, all the secondary regions of interest may be used, but the index may be calculated by selectively using either the linear scan image or the sector scan image depending on the part of the muscle.

The linear scan images and sector scan images have the following advantages and disadvantages. The linear scan image has the advantage of being less influenced by the characteristics of the echo and having high reliability. However, the linear scan image has the following disadvantages. Since the observation range at a given time is narrow and each secondary region of interest is only partially reflected, the linear scan image is easily affected by unevenness in the secondary region of interest. It is difficult to distinguish an image having a correct value from an image to be removed due to unevenness. In particular, the latter disadvantage is prominent in a linear scan image of the vastus medialis. In addition, the vastus medialis muscle has a smaller variation range than other muscles (with low sensitivity), and the effect of the directionality to which ultrasonic waves hit is greater.

On the other hand, the sector scan image has the following advantages. Since an observation range at a given time is wide and many images include all or most part of the secondary region of interest, the sector scan image is averaged even if there is unevenness in the secondary region of interest. It is easy to distinguish an image having a correct value from an image having an abnormal value (when the secondary region of interest is not reflected behind the bone). The vastus medialis muscle, which shows a large change in the linear scan image, shows the same change as other muscles in the sector scan image. On the other hand, the sector scan image is easily affected by the characteristics of the echo, and has a disadvantage that the deeper the sector scan image is, the worse the echo characteristics become.

Based on the characteristics of the linear scan image and the sector scan image as described above, an image for calculating the index may be selected for each muscle region. For the vastus lateralis and vastus intermedius in the quadriceps femoris 92, the correlation between the brightness of the secondary region of interest and the equivalent CT value tends to be higher in the linear scan image than in the sector scan image. Therefore, for the vastus lateralis and vastus intermedius, an index may be calculated using the linear scan image. On the other hand, for the rectus femoris and vastus medialis in the quadriceps femoris 92, the correlation between the brightness of the secondary region of interest and the CT value equivalent tends to be higher in the sector scan image than in the linear scan image. Therefore, for the rectus femoris and vastus medialis, an index may be calculated using the sector scan image.

The brightness can be expressed in 0-255 gradations. However, due to a possibility that an error may occur depending on the region of the muscle or the method of scanning, a brightness correction technique may be used to calculate a corrected brightness. The calculation of the corrected brightness is possible by a) correcting for scaling according to the part in order to evaluate a tendency of the part that is bright and the part that is dark appears in the same order, and b) correcting for differences of trends when using a mix of results from different scanning methods. As a result, it is not necessary to standardize settings related to image quality and brightness such as a gain of the transmitter/receiver 2.

Since the brightness is an image processing value under a specific condition, it can be generalized by converting it into a physical index. For example, brightness can be converted to dB as an echo reflection level. Specifically, a reference can be determined by a substance corresponding to 0 dB (For example, a phantom for quality control), and the brightness can be converted to dB from measurement or calculation. This makes it possible to correct variations in mass production through inspection and correction, and to standardize indices for multiple models.

Further, the index calculation module 324 may calculate a CT value equivalent value (HU), an MRI equivalent value (T1, T2), or a muscle tissue evaluation value (Fat equivalent (%), effective muscle cross-sectional area, etc.) of the image as an index corresponding to the muscle quality, instead of the average value of the brightness. These values can be correlated with clinically significant data, such as CT and MRI measurements and the actual anatomic and medical condition of the muscle.

The muscle quality evaluation module 325 shown in FIG. 4 evaluates the muscle quality based on the index calculated by the index calculation module 324. When the index is an average value of brightness, it can be evaluated that the larger the average value is, the larger the amount of fat in the muscle and lower is the muscle quality.

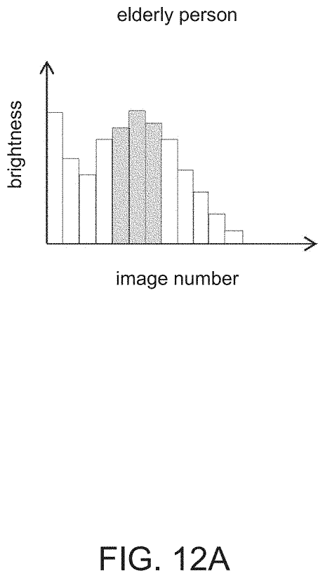

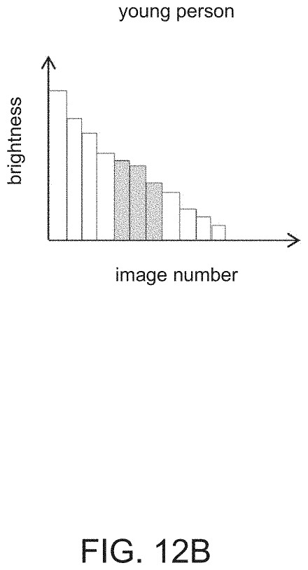

A histogram representing the brightness distribution can also be used to evaluate the muscle quality. For example, as shown in FIGS. 12A and 12B, since a ratio of the number of pixels to a specific brightness value is different between the elderly and the young, the muscle quality can be evaluated based on the ratio.

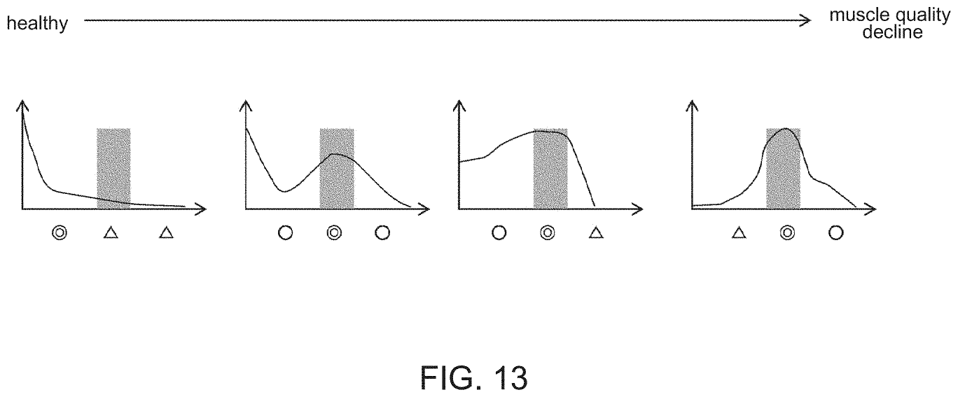

Alternatively, the muscle quality can be evaluated by a size relationship of the region of the brightness distribution. Since the brightness distribution differs as the muscle quality decreases from the healthy state, for example, as shown in FIG. 13, the brightness values in the gray area and the areas before and after the gray area are judged in 3 stages of ⊚ (abound), ◯ (intermediate), and Δ (little), and the muscle quality is evaluated by the combination of the size relation. In each graph of FIG. 13, the vertical axis and the horizontal axis represent brightness and image number, respectively.

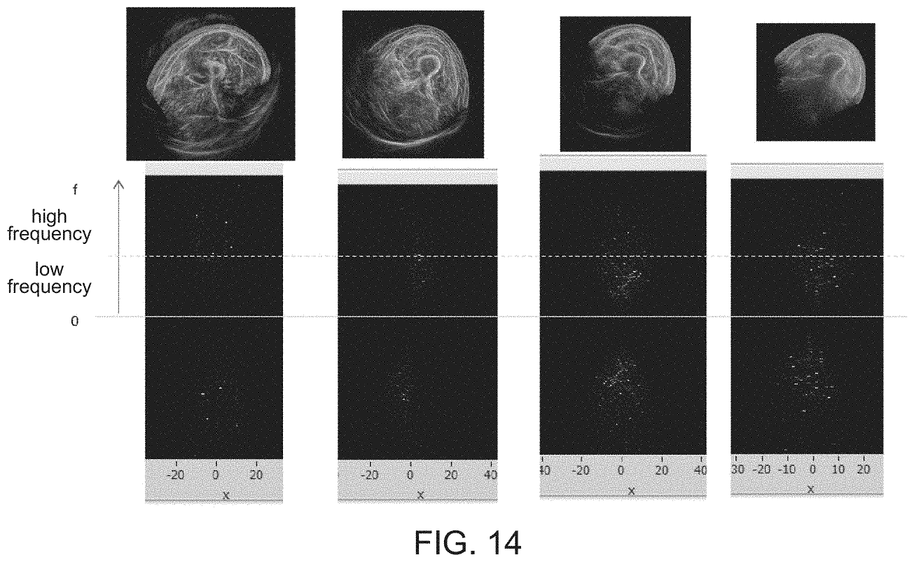

Alternatively, patterns and spatial frequencies can be used to evaluate muscle quality. For example, a two-dimensional fast Fourier transform (FFT: Fast Fourier Transform) is performed on a linear scan image. The upper four images shown in FIG. 14 are images before conversion, and the lower four images are images after conversion. In the normal state, a high frequency component of the brightness in the converted image is larger than a low frequency component, and the low frequency component is at a low level, but the distribution of the brightness moves to the low frequency side with the lowering of the muscle quality. Therefore, the muscle quality can be evaluated based on the high frequency component and the low frequency component. It should be noted that this evaluation method can improve the evaluation accuracy by additionally performing the above-mentioned muscle quality evaluation using the average value or histogram of brightness.

The cross-sectional area calculator 326 shown in FIG. 4 calculates the cross-sectional areas of the rectus femoris, vastus lateralis, vastus intermedius, and vastus medialis of the quadriceps femoris 92. The calculation result is an input to the muscle quality evaluation module 325 together with the evaluation result of the decision module 327. The decision module 327 also receives subject information T such as the age, sex, height, and weight of the body 9 stored in the recording module 34. The cross-sectional area calculator 326 and the subject information T may have any configuration.

The decision module 327 presents the evaluation result of the muscle quality, the muscle cross-sectional area, and reference data based on the subject information T inputted from the muscle quality evaluation module 325 and the cross-sectional area calculator 326, respectively. Further, the decision module 327 performs calculation (e.g., T-SCORE) in comparison with the reference data for determination. These results are output to the output module 33 and can be confirmed by the user.

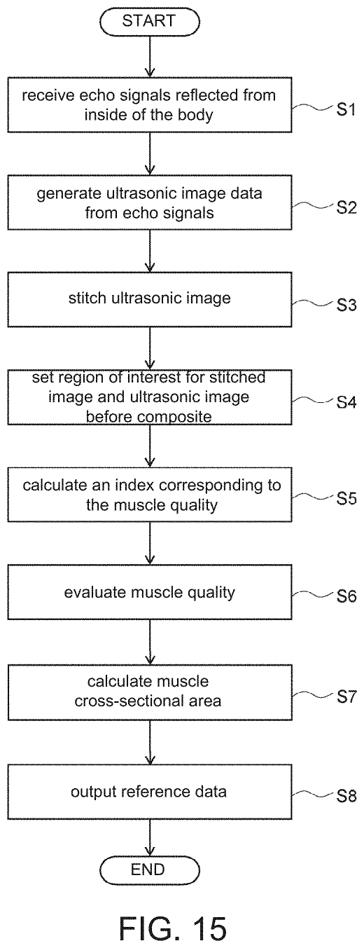

FIG. 15 is a flow chart showing a flow of processing in an ultrasonic analysis method according to the present embodiment. The ultrasonic analysis method according to the present embodiment can be implemented by the transmitter/receiver 2 and the ultrasonic analysis apparatus 3.

In step S1, the transmitter/receiver 2 shown in FIG. 1 transmits ultrasonic waves from different positions from the transducer of the ultrasonic probe 21 toward the muscles inside of the body 9, and receives the echo signals reflected from inside of the body 9 by the transducer of the ultrasonic probe 21. In this embodiment, the muscle is the quadriceps femoris 92. The transmitter/receiver 2 digitally converts the received echo signal and transmits it to the ultrasonic analysis apparatus 3, and the I/F module 35 of the ultrasonic analysis apparatus 3 receives the echo signal from the transmitter/receiver 2.

In step S2, the ultrasonic image generating module 321 shown in FIG. 4 generates an ultrasonic image of the inside of the body 9 based on the received echo signal. In the present embodiment, the ultrasonic image generating module 321 generates a linear scan image and a sector scan image as ultrasonic images.

In step S3, the image compositing module 322 stitches the ultrasonic images to generate a stitched image.

In step S4, the area setting module 323 sets a region of interest for the stitched image and the ultrasonic image before the stitching. In this embodiment, a region of interest is set for each of the rectus femoris, vastus lateralis, vastus intermedius, and vastus medialis constituting the quadriceps femoris 92.

In step S5, the index calculation module 324 calculates an index corresponding to the muscle quality based on the image corresponding to the secondary region of interest set for the ultrasonic image before the stitching. The index is, for example, an average value of brightness of an image.

In step S6, the muscle quality evaluation module 325 evaluates the muscle quality based on the index. The evaluation of the muscle quality can be performed based on the average value of the brightness, the histogram of the brightness, the ratio of the number of pixels of a specific brightness value, etc.

In step S7, the cross-sectional area calculating module 326 calculates a muscle cross-sectional area. In the present embodiment, the cross-sectional area calculation module 326 calculates the cross-sectional areas of the rectus femoris, vastus lateralis, vastus intermedius, and vastus medialis of the quadriceps femoris 92.

In step S7, the decision module 327 presents the evaluation result of the muscle quality, the muscle cross-sectional area, and reference data based on the subject information T.

According to the ultrasonic analysis apparatus 3 and the ultrasonic analysis method according to the present embodiment, it is possible to evaluate the muscle quality by analyzing an ultrasonic image without using a CT apparatus or an MRI apparatus having a complicated configuration as in the prior art. Therefore, it is possible to evaluate the muscle quality with a relatively simple constitution without accompanying exposure to possibly harmful radiations.

While the embodiments of the present invention have been described above, the present invention is not limited to the embodiments described above, and various modifications can be made without departing from the spirit thereof, and, for example, forms obtained by appropriately combining the technical means disclosed in the embodiments are also within the technical scope of the present invention.

In the above embodiment, the index calculation module 324 calculates the index corresponding to the muscle quality from the image corresponding to the region of interest set for the ultrasonic image before synthesis, but the index may be calculated from the image corresponding to the region of interest set for the stitched image. , the brightness decreases according to the depth in the ultrasonic image, and thus, the brightness is affected in the stitched image. Therefore, the index may be calculated from the image corresponding to the region of interest set for the ultrasonic image before synthesis.

Further, in the above embodiment, while one ultrasonic probe 21 is moved along the surface of the thigh 91, a plurality of ultrasonic images are acquired by transmitting ultrasonic waves from different positions toward the quadriceps femoris 92 a plurality of times, but the method for acquiring the plurality of ultrasonic images is not limited thereto. For example, a plurality of ultrasonic probes, similar to the ultrasonic probe 21, may be disposed around the thigh 91, and ultrasonic waves may be simultaneously transmitted from each ultrasonic probe toward the quadriceps femoris 92 to obtain the plurality of ultrasonic images.

Although the ultrasonic image generating module 321 generates the linear scan image and the sector scan image as ultrasonic images in the aforementioned embodiment, the ultrasonic image generating module 321 may generate only one of the linear scan image and the sector scan image in various other embodiments. Alternatively, the ultrasonic image generating module 321 may generate a convex image as an ultrasonic image. In this case, the region of interest may be set in the convex image, and the index corresponding to the muscle quality may be calculated from the image corresponding to the region of interest.

In the above embodiment, 200 linear scan images and 200 sector scan images are used for calculating the index respectively, but the number of ultrasonic images used for calculating the index is not particularly limited, and a part of the acquired ultrasonic images may be selected and used for calculating the index. For example, 50 images may be selected at predetermined intervals from the total 400 ultrasonic images arranged in an order of acquisition.

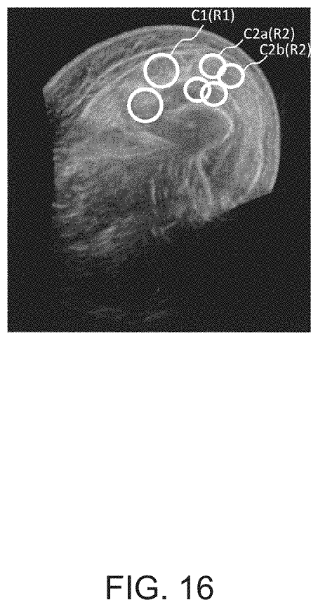

Further, when setting the region of interest in the stitched image, in the above embodiment, the quadriceps femoris 92 is divided into four regions of the rectus femoris, vastus lateralis, vastus intermedius, and vastus medialis, but it may be difficult to manually find the boundary of the region depending on the subject. Therefore, for example, as shown in FIG. 16, a typical position of the muscle may be surrounded by a frame having a simple shape such as a circular frame, and an area within the frame may be set as a region of interest. In the example shown in FIG. 16, a circular frame C1 including the rectus femoris is set as a region of interest R1. In the case of a muscle having an elongated cross-sectional shape such as the vastus lateralis muscle, a region within the muscle may be surrounded by a plurality of circular frames C2a and C2b, and a region within the circular frames C2a and C2b may be set as a region of interest R2.

When the quadriceps femoris 92 is used as the analysis subject of the muscle quality, one region of interest may be set for the entire quadriceps femoris 92, or at least one of the rectus femoris, vastus lateralis, vastus intermedius, and vastus medialis constituting the quadriceps femoris 92.

In the above embodiment, the analysis subject of the muscle is the quadriceps femoris 92, but the present invention is not limited thereto, and any muscle such as the back of the thigh 91, the calf, and the upper arm muscle can be included in the analysis subject.

Further, in the above embodiment, in order to obtain an ultrasonic image corresponding to a plurality of mutually different positions on the surface of the body 9, the ultrasonic wave is intermittently transmitted from the ultrasonic probe 21 while the ultrasonic probe 21 is moved along the surface of the body 9, the embodiments for obtaining ultrasound images are not limited thereto. For example, a plurality of ultrasonic probes 21 may be arranged in the body 9, and ultrasonic waves may be transmitted simultaneously from each ultrasonic probe 21.

### TERMINOLOGY

Not all purposes or benefits may be achieved in accordance with any particular embodiment described herein. Accordingly, those skilled in the art will appreciate, for example, that certain embodiments may be configured to operate to achieve or optimize one or more of the effects or advantages as taught herein without necessarily achieving other purposes or advantages as taught or suggested herein.

All processing described herein may be embodied and fully automated by a software code module executed by a computing system including one or more computers or processors. The code module may be stored on any type of non-temporary computer readable medium or other computer storage device. Some or all of the methods may be embodied in dedicated computer hardware.

It will be apparent from the present disclosure that there are many other variations other than those described herein. For example, depending on the embodiment, any particular operation, event, or function of the algorithms described herein may be performed in a different sequence and may be added, merged, or totally excluded (For example, not all of the actions or events described are necessary for the execution of the algorithm). Furthermore, in certain embodiments, operations or events may be executed in parallel, rather than sequentially, e.g., via multi-threading, interrupt processing, or multiple processors or processor cores, or on other parallel architectures. In addition, different tasks or processes may be performed by different machines and/or computing systems that may work together.

The various exemplary logic blocks and modules described in connection with the embodiments disclosed herein may be implemented or executed by a machine, such as a processor. The processor may be a microprocessor, or alternatively, the processor may be a controller, a microcontroller, a state machine, or a combination thereof. The processor may include electrical circuitry configured to process computer-executable instructions. In another embodiment, the processor includes an application specific integrated circuit (ASIC), a field programmable gate array (FPGA), or other programmable device that performs logic operations without processing computer executable instructions. The processor may also be implemented as a combination of computing devices, such as a combination of a digital signal processor (Digital signal processor) and a microprocessor, a plurality of microprocessors, one or more microprocessors in combination with a DSP core, or any other such configuration. Although described herein primarily with respect to digital techniques, the processor may also primarily comprise analog elements. For example, some or all of the signal processing algorithms described herein may be implemented by an analog circuit or a mixed analog and digital circuit. The computing environment may include any type of computer system, including, but not limited to, a microprocessor, a mainframe computer, a digital signal processor, a portable computing device, a device controller, or a computer system based on a computing engine within the device.

Unless otherwise specified, conditional languages such as “capable” “I did.” “Yeah.” or “There is a possibility.” are understood within the context generally used to convey that a particular embodiment includes particular features, elements and/or steps, but not other embodiments. Thus, such conditional language does not generally mean that the features, elements and/or steps are in any manner required by one or more embodiments, or that the one or more embodiments necessarily include logic for determining whether these features, elements and/or steps are included in or performed by any particular embodiment.

A disjunctive language such as the phrase “At least one of X, Y, Z” is understood in the context in which it is generally used to indicate that an item, term, etc. may be X, Y, Z, or any combination thereof (Example: X, Y, Z), unless otherwise stated. Thus, such a disjunctive language generally does not mean that a particular embodiment requires each of at least one of X, at least one of Y, or at least one of Z, each being present.

Any process description, element, or block in the flow diagram described herein and/or shown in the accompanying drawings should be understood to represent a potentially module, segment, or portion of code that includes one or more executable instructions for implementing a particular logic function or element in the process. Alternative embodiments are included within the scope of the embodiments described herein, wherein the elements or functions may be removed from those shown or described, in random order, substantially simultaneously or in reverse order, depending on the associated functionality, as will be understood by those skilled in the art.

Unless otherwise specified, a numeral such as “one” should generally be interpreted to include one or more stated items. Thus, the term “one device configured to ˜” is intended to include one or more enumerated devices. Such one or more enumerated devices may also be collectively configured to perform the recited citation. For example, the “Processor configured to run the following A, B, and C” may include a 1st processor configured to execute A and a 2nd processor configured to execute B and C. In addition, even if enumerations of specific numbers of the introduced embodiments are explicitly enumerated, those skilled in the art should interpret such enumerations as typically meaning at least the enumerated numbers (For example, a simple enumeration of “With the two enumerations” without other modifiers typically means at least 2 enumerations, or 2 or more enumerations).

In general, it will be appreciated by those skilled in the art that the terms used herein are generally intended to be the “Unlimited” term (For example, the term “inclusive of” should be interpreted as “plus at least ˜”, the term “to have ˜” should be interpreted as “have at least ˜”, the term “Include” should be interpreted as “including, but not limited to, the following:”, etc.).

For purposes of description, the term “HORIZONTAL” as used herein, regardless of direction, is defined as a plane parallel to the floor or surface of the area in which the described system is used, or a plane in which the method described is performed. The term ‘floor’ may be replaced by the term “ground” or “water surface”. The term “Vertical/Vertical” refers to a direction perpendicular/perpendicular to the defined horizon. Terms such as “upper side”, “lower side”, “side”, “higher”, “lower”, “upwards”, “beyond” and “lower” are defined relative to the horizontal plane.

The terms “attach”, “connect”, “pair up” and other related terms as used herein are to be interpreted to include removable, movable, fixed, adjustable, and/or removable connections or linkages, unless otherwise noted. The connection/connection includes a direct connection and/or a connection having an intermediate structure between the two components described.

Unless expressly stated otherwise, the numbers preceded by terms such as “approximately”, “in substance”, and the like, as used herein, include the enumerated numbers and represent quantities close to the stated quantities performing a desired function or achieving a desired result. For example, “approximately”, “in substance” and “about” refer to values less than 10% of the stated values, unless otherwise indicated. As used herein, a feature of an embodiment in which terms such as “approximately”, “in substance” and the like are previously disclosed further represents a feature having some variability in performing a desired function or achieving a desired result with respect to that feature.

Many variations and modifications can be made to the embodiments described above, and those elements should be understood as being within other acceptable examples. All such modifications and variations are intended to fall within the scope of the present disclosure and are protected by the following claims.

What is claimed is:

1. An ultrasonic analysis apparatus, comprising:
an interface configured to receive signals from a transmitter/receiver which receives ultrasonic waves transmitted from a plurality of mutually different positions on a surface of a body toward a muscle inside the body and reflected from inside the body; and
processing circuitry configured
to generate a plurality of ultrasonic images corresponding to the respective positions based on the signals inputted from the interface;
to stitch the plurality of ultrasonic images at the respective positions to generate a stitched image;
to set a region of interest for at least one of the stitched image and an ultrasonic image of the plurality of ultrasonic images; and
to calculate an index related to the muscle from at least one of the images corresponding to the region of interest.

2. The ultrasonic analysis apparatus of claim 1, wherein:
the processing circuitry calculates the index from an image corresponding to the region of interest set for the ultrasonic image.

3. The ultrasonic analysis apparatus of claim 1, wherein:
the plurality of ultrasonic images include at least one of a linear scan image and a sector scan image.

4. The ultrasonic analysis apparatus of claims 1, wherein:
the index is corresponding to a muscle quality of the muscle.

5. The ultrasonic analysis apparatus of claim 1, wherein:
the index is an average value of a brightness of the image corresponding to the region of interest.

6. The ultrasonic analysis apparatus of claim 1, wherein:
the muscle is a quadriceps femoris.

7. The ultrasonic analysis apparatus of claim 6, wherein:
the processing circuitry is further configured:
to set the region of interest for each of a rectus femoris, a vastus lateralis, a vastus intermedius, and a vastus medialis of the quadriceps femoris; and
to calculate the index related to each muscle of the rectus femoris, the vastus lateralis, the vastus intermedius, and the vastus medialis.

8. The ultrasonic analysis apparatus of claim 7, wherein:
the plurality of ultrasonic images include a sector scan image and a linear scan image, and
the processing circuitry calculates the index related to each muscle of the vastus lateralis and the vastus intermedius from an image corresponding to the region of interest set in the linear scan image, and calculates the index related to each muscle of the rectus femoris and the vastus medialis from an image corresponding to the region of interest set in the sector scan image.

9. The ultrasonic analysis apparatus of claim 1, wherein:
the processing circuitry extracts a mask image comprising the region of interest set in the stitched image from the stitched image and sets the region of interest for the ultrasonic image by overlapping the mask image on the ultrasonic image.

10. An ultrasonic analysis method, comprising:
receiving signals from a transmitter/receiver which receives ultrasonic waves transmitted from a plurality of mutually different positions on a surface of a body toward a muscle inside the body and reflected from inside the body;
generating a plurality of ultrasonic images corresponding to the respective positions based on the received signals;
stitching the plurality of ultrasonic images at the respective positions to generate a stitched image;
setting a region of interest for at least one of the stitched image and an ultrasonic image of the plurality of ultrasonic images; and
calculating an index related to the muscle from at least one of the images corresponding to the region of interest.

11. A non-transitory computer-readable recording medium storing a program causing a processor of an ultrasound analysis apparatus to execute processing, the processor configured to control operation of the ultrasound analysis apparatus, the processing comprising:
receiving signals from a transmitter/receiver which receives ultrasonic waves transmitted from a plurality of mutually different positions on a surface of a body toward a muscle inside the body and reflected from inside the body;
generating a plurality of ultrasonic images corresponding to the respective positions based on the received signals;
stitching the plurality of ultrasonic images at the respective positions to generate a stitched image;
setting a region of interest for at least one of the stitched image and an ultrasonic image of the plurality of ultrasonic images; and
calculating an index related to the muscle from at least one of the images corresponding to the region of interest.

12. The ultrasonic analysis apparatus of claim 2, wherein:
the plurality of ultrasonic images include at least one of a linear scan image and a sector scan image.

13. The ultrasonic analysis apparatus of claim 2, wherein:
the index is corresponding to a muscle quality of the muscle.

14. The ultrasonic analysis apparatus of claim 2, wherein:
the index is an average value of a brightness of the image corresponding to the region of interest.

15. The ultrasonic analysis apparatus of claim 2, wherein:
the muscle is a quadriceps femoris.

16. The ultrasonic analysis apparatus of claim 2, wherein:
the processing circuitry extracts a mask image comprising the region of interest set in the stitched image from the stitched image and sets the region of interest for the ultrasonic image by overlapping the mask image on the ultrasonic image.

17. The ultrasonic analysis apparatus of claim 3, wherein:
the index is corresponding to a muscle quality of the muscle.

18. The ultrasonic analysis apparatus of claim 3, wherein:
the index is an average value of a brightness of the image corresponding to the region of interest.

19. The ultrasonic analysis apparatus of claim 3, wherein:
the muscle is a quadriceps femoris.

20. The ultrasonic analysis apparatus of claim 3, wherein:
the processing circuitry extracts a mask image comprising the region of interest set in the stitched image from the stitched image and sets the region of interest for the ultrasonic image by overlapping the mask image on the ultrasonic image.
